(19) United States
(12) Patent Application Publication
(10) Pub. No.: US 2025/0208067 A1
(43) Pub. Date: Jun. 26, 2025

(54) RADIATION IMAGING APPARATUS, RADIATION IMAGING METHOD, AND STORAGE MEDIUM

(71) Applicant: CANON KABUSHIKI KAISHA, Tokyo (JP)

(72) Inventor: TAKANORI TAYA, Kanagawa (JP)

(21) Appl. No.: 18/979,046

(22) Filed: Dec. 12, 2024

(30) Foreign Application Priority Data
Dec. 21, 2023 (JP) 2023-216196

(51) Int. Cl. G01N 23/04; G01T 1/02

(52) U.S. Cl. CPC G01N 23/04; G01T 1/02; G01N 2223/304; G01N 2223/306

(57) ABSTRACT

A radiation imaging apparatus includes a plurality of receptor fields where a dose of radiation capable of being detected during radiation imaging in which a radiation irradiation apparatus emits the radiation. The radiation imaging apparatus is capable of executing processing for stopping the irradiation based on a result of the dose detection in at least one of the plurality of receptor fields. The radiation imaging apparatus includes one or more controllers configured to adjust a parameter associated with the receptor field used for the dose detection based on registration error information of an object at the time of the radiation imaging.

### BACKGROUND OF THE INVENTION

### Field of the Invention

The disclosed technique relates to a radiation imaging apparatus, a radiation imaging method, and a storage medium.

### Description of the Related Art

As an imaging apparatus used for medical imaging diagnosis or non-destructive inspection by X-rays, a radiation imaging apparatus formed by combining a conversion element that converts radiation into an electric charge, a switch element such as a thin film transistor, a pixel array provided with a wiring, a driving circuit, and a readout circuit has currently been put into practical use.

One of the radiation imaging apparatuses has a function of detecting irradiation information while a radiation source emits radiation. This function includes a function of detecting an incident start timing at which the radiation source emits radiation and a function of detecting the dose and integrated dose of the radiation. With this function, it is possible to execute automatic exposure control in which the integrated dose is monitored and a detection apparatus controls the radiation source to end irradiation when the integrated dose reaches an appropriate amount.

Japanese Patent Laid-Open No. 2021-79023 discloses a technique of performing, with an appropriate dose, imaging of a part to be captured by controlling irradiation with radiation based on information associated with a region of interest when automatic exposure control is used.

However, in the technique disclosed in Japanese Patent Laid-Open No. 2021-79023, since a detection region where an integrated dose is detected is at a fixed position, it is necessary to select a detection region in advance in accordance with an imaging part of an object. In this case, if the detection region selected in advance deviates from the position of the object, or a difference in body thickness occurs due to a difference in age/sex and the like, it is impossible to appropriately control exposure, and thus the dose of radiation may vary.

The disclosed technique provides a radiation imaging technique capable of suppressing the variation of the dose of radiation.

### SUMMARY OF THE INVENTION

According to one aspect of the present invention, there is provided a radiation imaging apparatus that includes a plurality of receptor fields where a dose of radiation capable of being detected during radiation imaging in which a radiation irradiation apparatus emits the radiation and is capable of executing processing for stopping the irradiation based on a result of the dose detection in at least one of the plurality of receptor fields, the radiation imaging apparatus comprising: one or more controllers configured to adjust a parameter associated with the receptor field used for the dose detection based on registration error information of an object at the time of the radiation imaging.

According to another aspect of the present invention, there is provided a radiation imaging method of a radiation imaging apparatus that includes a plurality of receptor fields where a dose of radiation capable of being detected during radiation imaging in which a radiation irradiation apparatus emits the radiation and is capable of executing processing for stopping the irradiation based on a result of the dose detection in at least one of the plurality of receptor fields, the radiation imaging method comprising: adjusting a parameter associated with the receptor field used for the dose detection based on registration error information of an object at the time of the radiation imaging.

Further features of the present invention will become apparent from the following description of exemplary embodiments (with reference to the attached drawings).

### BRIEF DESCRIPTION OF THE DRAWINGS

FIG. 1 is a view showing the configuration of a radiation imaging system according to the first embodiment;

FIG. 2 is a block diagram showing the arrangement of an FPD processing unit according to the first embodiment;

FIG. 3 indicates views showing examples of the variation of automatic exposure control in a case where the position of an object deviates in the up-and-down direction with respect to a radiation imaging apparatus according to the first embodiment;

FIGS. 4A and 4B are views showing examples of weighting based on output information in a case where the position of the object deviates upward with respect to the radiation imaging apparatus according to the first embodiment;

FIG. 5 indicates views showing an example of weighting based on an output ratio in a case where the position of the object deviates rightward according to the first embodiment;

FIG. 6 indicates a view and graphs showing an example of weighting based on output information in a case where the size of an object is small according to the second embodiment;

FIG. 7 indicates graphs each showing an example of a histogram in a case where a registration error of the position of an object 105 occurs with respect to an FPD 102;

FIG. 8 is a view showing the configuration of a radiation imaging system according to the second embodiment;

FIG. 9 is a view exemplifying a table that stores imaging condition information, reference output information, and reference weighting information;

FIG. 10 is a view exemplifying a table that stores reference physique information, reference output information, and reference weighting information; and

FIG. 11 is a view showing the configuration of a radiation imaging system according to the third embodiment.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

Note that each embodiment to be described below will explain a case where the disclosed technique is applied to an X-ray imaging apparatus, as a radiation imaging apparatus, that captures X-ray image data of an object using X-rays as a type of radiation. Furthermore, the disclosed technique is not limited to the X-ray imaging apparatus, and can be applied to a radiation imaging apparatus that captures a radiation image of an object using other types of radiation (for example, α-rays, β-rays, and γ-rays).

Auto Exposure Control (AEC) will mainly be described but the disclosed technique may be used for radiation dose measurement (monitoring) used for AEC and the imaging apparatus itself need not perform radiation control. In addition, the disclosed technique may be used to detect the start of irradiation with radiation and may further be used to detect the end of irradiation with radiation.

### First Embodiment

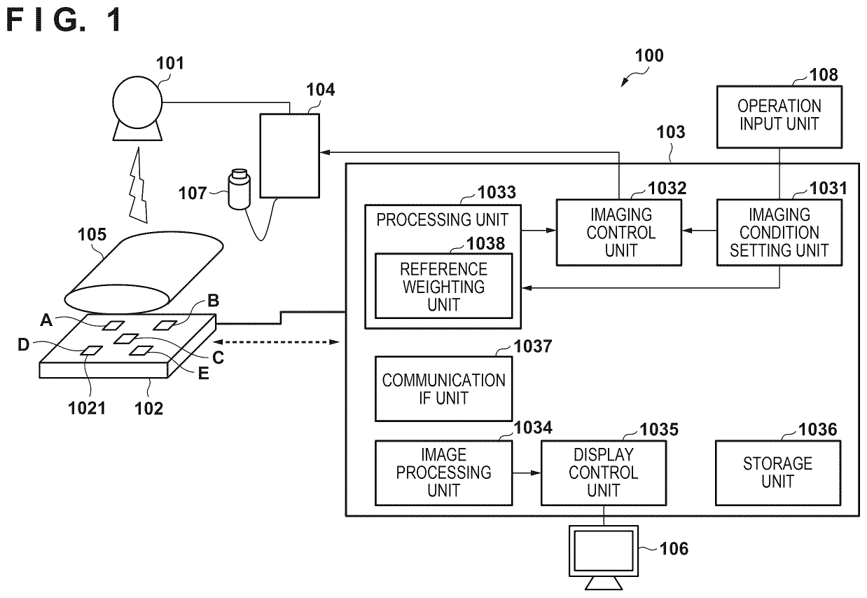

FIG. 1 is a view showing an example of the configuration of a radiation imaging system 100 including a radiation imaging apparatus according to the first embodiment. The radiation imaging system 100 is used at the time of, for example, capturing a radiation image in a hospital, and includes, as a system configuration, a radiation imaging apparatus 102 that captures a radiation image based on radiation emitted from a radiation source 101, and an imaging control apparatus 103. The imaging control apparatus 103 is connected to the radiation imaging apparatus 102 and a radiation control apparatus 104 that controls the radiation source 101 by, for example, a wired or wireless network or dedicated lines, and controls radiation imaging using the radiation imaging apparatus 102 and the radiation source 101.

### (Radiation Source 101)

Referring to FIG. 1, the radiation source 101 holds, for example, a rotor and an X-ray tube that accelerates electrons by a high voltage and collides them against an anode in order to generate radiation. The radiation source 101 irradiates an object 105 with X-rays.

### (Radiation Imaging Apparatus 102)

The radiation imaging apparatus 102 is a Flat Panel Detector (to be referred to as an FPD hereinafter) in which a plurality of pixels are arranged in a matrix on a flat substrate, and includes two-dimensionally distributed image sensors. The FPD 102 detects a two-dimensional distribution (dose information) of the doses of radiation that has been transmitted through the object 105 to reach the image sensors, thereby generating image data. The FPD 102 transmits the generated image data (radiation image data) to an image processing unit 1034 of the imaging control apparatus 103. Furthermore, the FPD 102 transmits, to the imaging control apparatus 103, the dose information of the detected two-dimensional distribution of the doses of radiation, and determination information for controlling irradiation with radiation in automatic exposure control.

In the FPD 102, detection pixels each including a radiation detection element for monitoring the dose of radiation are arranged. The detection pixels are distributed and arranged in the FPD 102. A detection region 1021 is arranged in the FPD 102, and includes a plurality of image data generation pixels and a plurality of detection pixels. The dose of radiation is monitored for each detection region, and a representative value is used as pixel information of the detection pixels in each detection region. As the pixel information (representative value) of the detection pixels in each detection region, an average value of signals of the detection pixels will be exemplified in the following embodiments. However, the present invention is not limited to this. The pixel information (representative value) of the detection pixels may be, for example, a median, a mode, an integrated value, or the like based on the arithmetic processing of signals detected by the plurality of detection pixels.

Furthermore, in a description of each of the following embodiments, the detection regions will be exemplified using an example of five regions or an example of nine regions but are not limited to these examples. In automatic exposure control (AEC) according to the disclosed technique, arithmetic processing is performed for signal values (monitor signal values) acquired from the detection pixels in a selected detection region, and thus the position and size of the detection region, the number of selected detection regions, and the like have no influence.

### (Imaging Control Apparatus 103)

The imaging control apparatus 103 relays communication between the FPD 102 and the radiation control apparatus 104. A communication method in the imaging control apparatus 103 may be a wired communication method or a wireless communication method. The imaging control apparatus 103 includes an imaging condition setting unit 1031, an imaging control unit 1032, a processing unit 1033, the image processing unit 1034, a display control unit 1035, a storage unit 1036, and a communication IF unit 1037. The communication IF unit 1037 functions as a communication interface for transmitting/receiving data to/from the FPD 102 or the radiation control apparatus 104.

The storage unit 1036 can be formed by, for example, a storage medium including a storage device such as a Hard Disk Drive (HDD), a Solid State Drive (SSD), or an optical disk drive, or the like. The storage unit 1036 can store various kinds of information and data acquired from respective unit components, information about an object, image data obtained by capturing the object in the past, various kinds of computer programs for executing processing of the imaging control apparatus 103, and a table of reference weighting information set for each detection region.

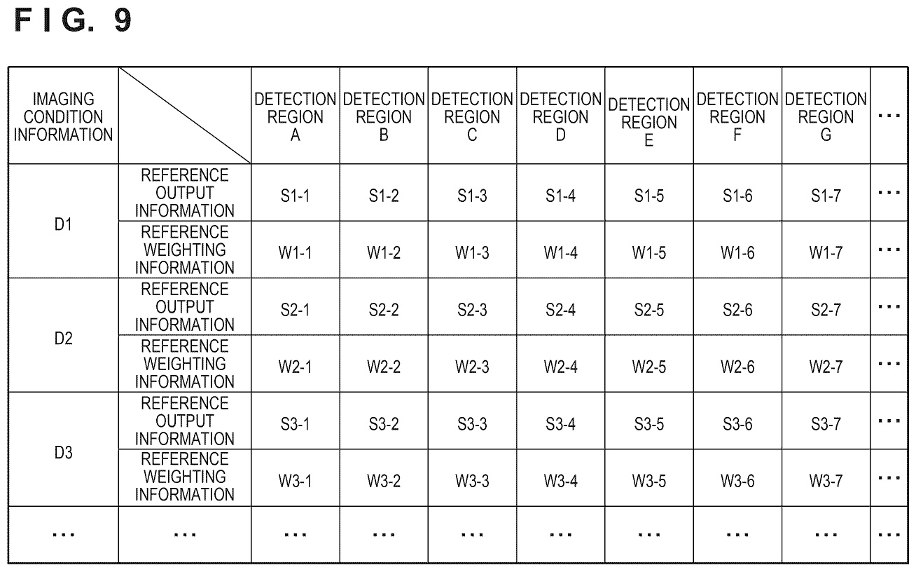

FIG. 9 is a view exemplifying a table that stores imaging condition information, reference output information, and reference weighting information. In the table of the reference weighting information, a reference value (reference output information) of an output signal in each detection region and reference weighting information in each detection region are set for each piece of imaging condition information. With reference to the table in the storage unit 1036, the reference value (reference output information) of the output signal in each detection region and the reference weighting information in each detection region, which are set for each piece of imaging condition information, can be acquired as initial values. The reference output information includes output signals output from a plurality of detection regions in a case where no registration error of the object occurs with respect to the plurality of detection regions. The reference weighting information includes a reference value of weighting information in a case where no registration error of the object occurs with respect to the plurality of detection regions.

The respective unit components of the imaging control apparatus 103 can function in accordance with computer programs. For example, the function of each unit component may be implemented by loading the computer program stored in the storage unit 1036 or the like and executing it by the processing unit 1033. Alternatively, some or all of the functions of the unit components of the imaging control apparatus 103 may be implemented by using dedicated circuits. A communication delay and a processing delay between the units in the imaging control apparatus 103 are managed in accordance with the communication method, communication contents, and processing contents. Therefore, each unit in the imaging control apparatus 103 can perform communication by expecting a communication delay and a processing delay.

The functional components of the imaging control apparatus 103 will be described next.

### (Imaging Condition Setting Unit 1031)

The imaging condition setting unit 1031 accepts imaging condition information input via an operation input unit 108 by an operator, and transmits the received imaging condition information to the imaging control unit 1032 and the processing unit 1033. For example, the imaging condition information includes, with respect to an object to be imaged, object information including the age, sex, and physique, an imaging part of the object, a tube voltage, a tube current, an irradiation time, and threshold information for controlling signal output to the radiation source 101 to stop the radiation source 101 with a predetermined dose. In addition, the imaging condition information includes reference output information for each detection region used to control radiation irradiation and reference weighting information. The imaging condition information input by the operator can be stored in the storage unit 1036.

### (Processing Unit 1033)

The processing unit 1033 transmits, to the imaging control unit 1032, the dose information and the determination information for controlling irradiation with radiation in automatic exposure control, which have been transmitted from the FPD 102. The “dose information” generally represents a dose, which has reached the FPD 102, of the dose of radiation emitted from the radiation source 101, but dose information similar to this may be used.

The processing unit 1033 includes a reference weighting unit 1038 as a functional component. The reference weighting unit 1038 sets reference output information and reference weighting information for the detection region in the FPD 102 based on the imaging condition information set by the imaging condition setting unit 1031. In a case where there exist a plurality of detection regions in the pixel region of the FPD 102, the reference weighting unit 1038 sets reference output information and reference weighting information for each detection region.

When imaging condition information for imaging the object 105 is input from the imaging condition setting unit 1031, the reference weighting unit 1038 specifies imaging condition information corresponding to the input imaging condition information with reference to the table in the storage unit 1036. The reference weighting unit 1038 acquires, as initial values, the reference value (reference output information) of an output signal in each detection region and reference weighting information in each detection region based on the specified imaging condition information, and transmits them to the FPD 102 via the communication IF unit 1037.

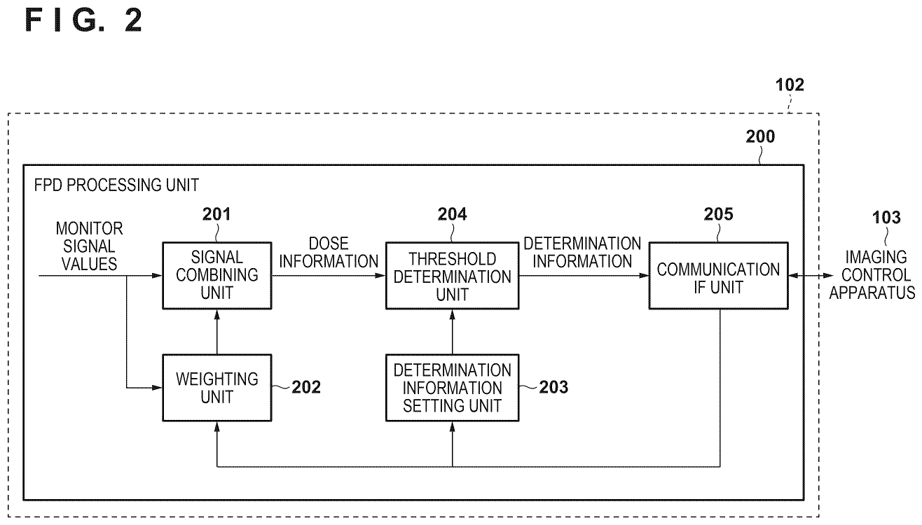

The FPD 102 includes an FPD processing unit 200 (FIG. 2) that processes the output signal output from the detection region, and a weighting unit 202 in the FPD processing unit 200 sets, as a reference value to be compared with an output signal (monitor signal value) output from each detection region in real time by imaging of the FPD 102, the reference value (reference output information) of the output signal transmitted from the reference weighting unit 1038.

The weighting unit 202 sets, as the initial value of the weighting information of each detection region, the reference weighting information transmitted from the reference weighting unit 1038. Based on comparison between the output signal (monitor signal value) output from each detection region in real time and the reference value (reference output information) of the output signal, the weighting unit 202 changes the reference weighting information set as the initial value. Practical processing of the weighting unit 202 will be described in detail later.

### (Imaging Control Unit 1032)

The imaging control unit 1032 controls the radiation control apparatus 104 and the FPD 102 based on the imaging condition information received from the imaging condition setting unit 1031 and the information received from the processing unit 1033.

### (Image Processing Unit 1034)

The image processing unit 1034 performs processing such as dark current correction, gain correction, defect correction, tone processing, and noise reduction processing for the radiation image data transmitted from the FPD 102. The image processing unit 1034 transmits the radiation image data having undergone the image processing to the display control unit 1035.

### (Display Control Unit 1035)

The display control unit 1035 performs display control of displaying the image information transmitted from the image processing unit 1034 on a display unit 106 such as a monitor. The display unit 106 is formed by, for example, an arbitrary device such as a Liquid Crystal Display (LCD), a Cathode Ray Tube (CRT) display, a plasma display panel, or an organic EL panel, and displays the radiation image data having undergone the image processing, which has been acquired from the image processing unit 1034.

### (Explanation of AEC Operation)

An AEC operation at the time of imaging an object will schematically be described next with reference to FIG. 1. Before imaging an object, imaging condition information including object information including the age, sex, and physique, an imaging part of the object, a tube voltage, a tube current, an irradiation time, and threshold information for controlling signal output to the radiation source 101 to stop the radiation source 101 with a predetermined dose is set with respect to the object to be imaged in the imaging condition setting unit 1031. At this time, reference output information and reference weighting information of the detection region used to control radiation irradiation may be input. The input imaging condition information may be sent to the imaging control unit 1032 and the processing unit 1033, and stored in the table in the storage unit 1036.

When an irradiation switch 107 mounted on the radiation control apparatus 104 is pressed, the radiation control apparatus 104 controls the radiation source 101 to start irradiation with radiation. When the cumulative dose of radiation reaches the predetermined dose after the radiation source 101 starts irradiation, the FPD 102 transmits determination information (an irradiation stop signal) to the imaging control apparatus 103, and the imaging control apparatus 103 transmits an irradiation stop signal to the radiation control apparatus 104. Upon receiving the irradiation stop signal, the radiation control apparatus 104 controls the radiation source 101 to stop irradiation with radiation from the radiation source 101.

At this time, the predetermined dose is a value calculated in consideration of the dose set before imaging the object, a change in X-ray irradiation intensity, and a communication delay and a processing delay between the units. If the irradiation time set before imaging the object is reached, the radiation control apparatus 104 stops irradiation with radiation from the radiation source 101 regardless of the presence/absence of an irradiation stop signal.

### (Functional Components of FPD Processing Unit 200)

Functional components associated with automatic exposure control (AEC) of the radiation imaging apparatus 102 (FPD) will be described next with reference to FIG. 2. The FPD processing unit 200 of the radiation imaging apparatus 102 is provided in the pixel region where a plurality pixels each for detecting radiation are arrayed, and processes output signals output from the plurality of detection regions each including the detection pixels each for outputting a signal corresponding to the dose of radiation. The FPD processing unit 200 includes, as functional components, a signal combining unit 201, the weighting unit 202, a determination information setting unit 203, a threshold determination unit 204, and a communication IF unit 205. The communication IF unit 205 functions as a communication interface for transmitting/receiving data to/from the imaging control apparatus 103.

### (Signal Combining Unit 201)

The signal combining unit 201 receives output signals (to be referred to as monitor signal values hereinafter) output from each detection region (for example, the detection region 1021 in FIG. 1 or the like), and outputs a monitor signal value (to be referred to as a combined monitor signal value hereinafter) obtained by performing signal combining processing for the received monitor signal values. The monitor signal value (combined monitor signal value) may be, for example, the average value of the detection pixel signal values included in each region or the average value of a predetermined number of detection pixel signal values selected from the plurality of detection pixels included in the detection region.

### (Weighting Unit 202)

The weighting unit 202 sets, as a reference value to be compared with an output signal (monitor signal value) output from each detection region in real time by imaging of the FPD 102, the reference value (reference output information) of the output signal transmitted from the reference weighting unit 1038 of the imaging control apparatus 103. Furthermore, the weighting unit 202 sets, as the initial value of the weighting information of each detection region, the reference weighting information transmitted from the reference weighting unit 1038.

The weighting unit 202 generates weighting information for each detection region by comparing the output signal (monitor signal value) output from each detection region in real time during imaging with the reference value (reference output information) of the output signal, and changes the reference weighting information set as the initial value.

The weighting unit 202 generates dose information based on the weighting information for each detection region and the monitor signal value (combined monitor signal value) output from the signal combining unit 201. As detailed processing, the weighting unit 202 generates dose information (weighted monitor signal value) by multiplying the monitor signal value (combined monitor signal value) by the weighting information for each detection region.

The weighting unit 202 may acquire the output information (reference output information) and the reference weighting information from the imaging control apparatus 103 via the communication IF unit 205, or may hold or generate reference output information and reference weighting information to generate weighting information for each detection region. For example, a plurality of pieces of output information (reference output information) as references may be held or generated in linkage with an imaging part, object information including the physique and age, and imaging condition information including a tube voltage and grid.

As the output information (reference output information), for example, the position of the object with respect to the FPD 102 (the plurality of detection regions) may be set as a reference or the ratio between the pieces of output information (reference output information) in the plurality of detection regions may be set as a reference. Alternatively, the information (reference physique information) set based on the physique of the object (for example, the width, thickness, and the like of the body of an adult or child) can be used as a reference. The weighting unit 202 may select, as representative pixels, a predetermined number of detection pixels from the plurality of detection pixels in the detection region, and performs a weighting operation for monitor signal values from the selected representative detection pixels. In this case, it is possible to reduce the load of the arithmetic processing, as compared with weighting for all the detection pixels in the detection region. Furthermore, the weighting unit 202 may further select a detection pixel at a necessary position from the selected detection pixels.

### (Determination Information Setting Unit 203)

The determination information setting unit 203 sets a threshold for the dose information representing a representative value for each detection region. Note that as the threshold set by the determination information setting unit 203, information included in the imaging condition information set by the imaging condition setting unit 1031 of the imaging control apparatus 103 and acquired via the communication IF unit 205 may be used. The set threshold is information used to determine whether the dose of emitted radiation reaches the cumulative dose in an AEC operation.

### (Threshold Determination Unit 204)

The weighting unit 202 of the FPD processing unit 200 generates weighting information for each of the plurality of detection regions by comparing the output signal with the preset reference information, and the threshold determination unit 204 generates determination information for controlling irradiation with radiation by comparing, with the preset threshold, the dose information obtained by weighting the output signal based on the generated weighting information. By comparing the dose information (weighted monitor signal value) generated by the weighting unit 202 with the threshold set by the determination information setting unit 203, the threshold determination unit 204 determines whether the dose from the radiation source 101 reaches a predetermined cumulative dose. Then, based on the determination result, the threshold determination unit 204 generates determination information for controlling irradiation with radiation. The determination information is information (irradiation control information) used to control irradiation with radiation (continue irradiation or stop irradiation) in AEC. The determination information generated by the threshold determination unit 204 is transmitted to the imaging control unit 1032 via the communication IF unit 205.

If the dose information (monitor signal value×weighting information) is lower than the threshold before the irradiation time preset before imaging the object is reached, the determination information is information (an irradiation continuation signal) that instructs to continue irradiation with radiation, and if the dose information is equal to or higher than the threshold, the determination information is information (an irradiation stop signal) that instructs to stop irradiation with radiation. Note that if the irradiation time set before imaging the object is reached, the radiation control apparatus 104 stops irradiation with radiation from the radiation source 101 regardless of the presence/absence of an irradiation stop signal.

The threshold determination unit 204 may determine whether the dose has reached the predetermined cumulative dose by comparing the dose information with the threshold based on a determination method (AND condition, OR condition, AVG condition, or the like) represented by a logical expression.

In a case where the determination processing is performed based on the AND condition (logical product), the threshold determination unit 204 determines, based on the AND condition (logical product), whether all the pieces of dose information of the plurality of detection regions are equal to or higher than the threshold. If all the pieces of dose information of the plurality of detection regions are equal to or higher than the threshold, determination information (an irradiation stop signal) for stopping irradiation with radiation is generated and output. For example, if the dose information in the detection region, where the dose information is lowest, among the plurality of detection regions is equal to or higher than the threshold, an irradiation stop signal is generated and output. In the determination processing based on the AND condition (logical product), radiation imaging can be performed without shortage of the dose in all the plurality of detection regions.

Alternatively, in a case where the determination processing is performed based on the OR condition (logical sum), the threshold determination unit 204 determines, based on the OR condition (logical sum), whether any of the pieces of dose information of the plurality of detection regions is equal to or higher than the threshold. If any of the pieces of dose information of the plurality of detection regions is equal to or higher than the threshold, determination information (an irradiation stop signal) for stopping irradiation with radiation is generated and output.

For example, if the dose information in the detection region, where the dose information is highest, among the plurality of detection regions is equal to or higher than the threshold, an irradiation stop signal is generated and output. In the determination processing based on the OR condition (logical sum), radiation imaging can be performed while suppressing excessive irradiation with radiation.

In a case where the determination processing is performed based on the AVG condition (averaging), the threshold determination unit 204 determines, based on the AVG condition (averaging), whether dose information obtained by averaging the pieces of dose information of the plurality of detection regions is equal to or higher than the threshold. If the averaged dose information is equal to or higher than the threshold, determination information (an irradiation stop signal) for stopping irradiation with radiation is generated and output.

The determination method (AND condition, OR condition, AVG condition, or the like) represented by a logical expression may be set by default, and a radiographer may change the setting of the determination method. Alternatively, the FPD processing unit 200 may change the setting of the logical expression of the determination method in accordance with the imaging condition information set by the imaging condition setting unit 1031. Alternatively, the FPD processing unit 200 may change the setting of the determination method (AND condition, OR condition, AVG condition, or the like) in automatic exposure control (AEC) in accordance with comparison between the monitor signal value and the output information (reference output information) as a reference during imaging by the FPD 102.

The arrangement in which the functional components of the FPD processing unit 200 are provided in the FPD 102 and the FPD 102 executes the automatic exposure control (AEC) determination function has been explained with reference to FIG. 2 but the present invention is not limited to this. The functional components of the FPD processing unit 200 may be provided in the processing unit 1033 of the imaging control apparatus 103 and the imaging control apparatus 103 may execute the AEC determination function. In this case, the FPD 102 may transmit the monitor signal value of each detection region to the imaging control apparatus 103 via the communication IF unit 205, and the processing unit 1033 of the imaging control apparatus 103 may execute processing associated with the AEC determination function based on the received monitor signal value.

### (AEC Operation)

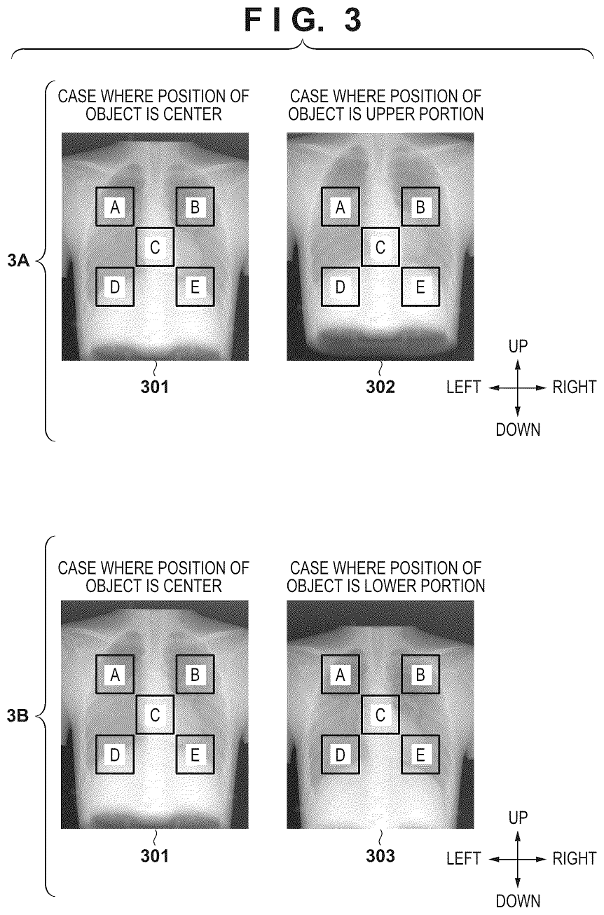

Automatic exposure control (AEC) in a case where a registration error of the object occurs with respect to the FPD 102, which is a problem for the radiation imaging apparatus 102 according to the first embodiment, will be described next with reference to 3A and 3B of FIG. 3. 3A and 3B of FIG. 3 are views showing examples of the variations of the monitor signal values in automatic exposure control in a case where the position of the object 105 deviates in the up-and-down direction of the paper surface with respect to the radiation imaging apparatus 102 (FPD) according to the embodiment. As for the detection region 1021, five detection regions A to E will be exemplified. Pieces of identification information (for example, A to E) are set in the detection regions, and each detection region can be identified based on the identification information. Among the five detection regions A to E exemplified here, the detection regions A and B are provided on the upper side (upper stage side) of the paper surface within the detection surface of the FPD 102. The detection regions D and E are provided on the lower side (lower stage side) of the paper surface within the detection surface of the FPD 102, as compared with the positions of the detection regions A and B. The position in the up-and-down direction of the detection region C is provided between the position of the detection regions A and B and the position of the detection regions D and E within the detection surface of the FPD 102. Furthermore, the position in the left-and-right direction of the detection region C is provided between the position of the detection regions A and D and the position of the detection regions B and E within the detection surface of the FPD 102.

In 3A of FIG. 3, 302 indicates a status in which the position of the object 105 deviates upward on the paper surface with respect to the FPD 102. At this time, the detection regions A to E of the FPD 102 are relatively located below the object 105.

In the detection region D or E located below (on the lower stage of) the detection regions A to C, since the ratio of the abdominal part with low radiation transmittance is high in the object structure in the detection region, the monitor signal value detected in the detection region D or E can decrease.

On the other hand, in the detection region A, B, or C, the object structure in the detection region does not change significantly. However, in the detection region A, B, or C, the ratio of the object structure with the same radiation transmittance increases, and thus the monitor signal value detected in the detection region A, B, or C can increase.

In a status in which the object 105 deviates upward on the paper surface with respect to the FPD 102, the monitor signal value in the detection region D or E can decrease, as compared with a case where the object 105 is located at the center of the FPD 102 (no relative registration error occurs: 301 in 3A of FIG. 3). Thus, if AEC is executed based on the monitor signal value in the detection region D or E, the time until the predetermined dose is reached becomes long, and the object 105 is unwantedly irradiated with more radiation than the predetermined dose. Alternatively, if AEC is executed based on the monitor signal value in the detection region A, B, or C, the time until the predetermined dose is reached becomes short, and the object 105 is irradiated with less radiation than the predetermined dose.

3B of FIG. 3 shows a status in which the position of the object 105 deviates downward on the paper surface with respect to the FPD 102. At this time, the detection regions A to E of the FPD 102 are relatively located above the object 105.

In the detection region D or E located below (on the lower stage of) the detection regions A to C, since the ratio of the abdominal part with low radiation transmittance is low in the object structure in the detection region, the monitor signal detected in the detection region D or E can increase.

On the other hand, in the detection region A, B, or C, the object structure in the detection region does not change significantly. However, in the detection region A, B, or C, the ratio of the object structure with the same radiation transmittance decreases, and thus the monitor signal value detected in the detection region A, B, or C can decrease.

In a status in which the object 105 deviates downward on the paper surface with respect to the FPD 102, the monitor signal value in the detection region D or E can increase, as compared with a case where the object 105 is located at the center of the FPD 102 (no relative registration error occurs). Thus, if AEC is executed based on the monitor signal value in the detection region D or E, the time until the predetermined dose is reached becomes short, and the object 105 is irradiated with less radiation than the predetermined dose. Alternatively, if AEC is executed based on the monitor signal value in the detection region A, B, or C, the time until the predetermined dose is reached becomes long, and the object 105 is unwantedly irradiated with more radiation than the predetermined dose.

As described above, since the monitor signal value changes due to a relative registration error between the object 105 and the FPD 102, the dose of the reference output (at the time of ideal imaging) varies.

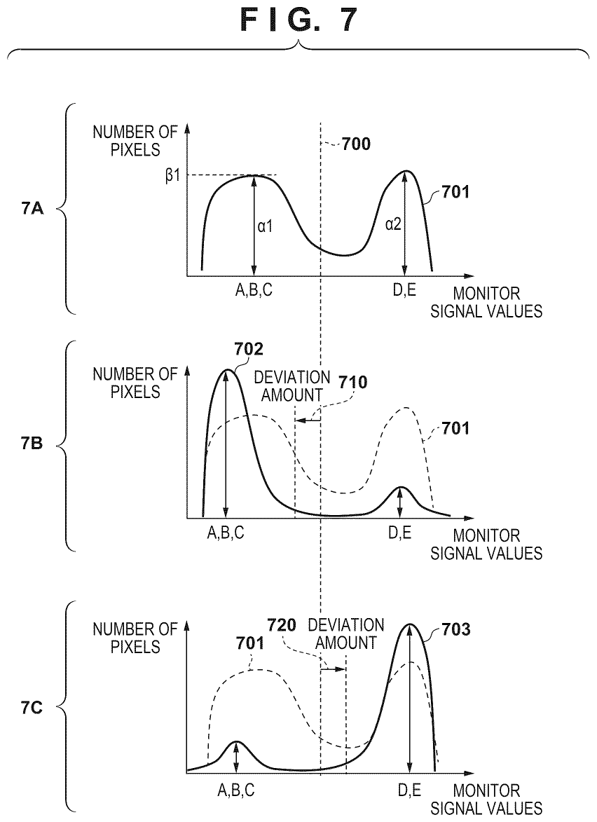

7A to 7C of FIG. 7 are graphs each showing an example of a histogram in a case where a registration error occurs in the position of the object 105 with respect to the FPD 102. A histogram 701 shown in 7A of FIG. 7 is a histogram indicating a state in which no registration error occurs. The histogram 701 is a histogram in the case 301, shown in 3A and 3B of FIG. 3, where the object 105 is located at the center of the FPD 102.

In this example, α1 represents output information (reference output information) as a reference in the detection region A, B, or C, and α2 represents output information (reference output information) as a reference in the detection region D or E. β1 represents the number of pixels of a detection region that outputs a monitor signal value corresponding to the reference output information α1. 7A of FIG. 7 shows a state in which no registration error of the object 105 occurs with respect to the FPD 102 (the plurality of detection regions), and the histogram has peaks in the reference output information α1 in the detection region A, B, or C and the reference output information α2 in the detection region D or E.

A histogram 702 shown in 7B of FIG. 7 corresponds to 302 of 3A of FIG. 3, and shows a status in which the position of the object 105 deviates upward with respect to the FPD 102. An arrow 710 in the histogram 702 indicates a deviation amount in the upper direction from a central portion 700 of the FPD 102.

Since the ratio of the abdominal part with low radiation transmittance is high in the object structure in the detection region D or E, the number of pixels in the detection region D or E that outputs a monitor signal value corresponding to the reference output information α2 can decrease.

On the other hand, since the ratio of the object structure with the same radiation transmittance increases in the detection region A, B, or C, the number of pixels in the detection region A, B, or C that outputs the monitor signal value corresponding to the reference output information α1 can increase. For example, if the threshold determination processing is performed based on the AND condition (logical product), the monitor signal value decreases, as compared with the assumed reference output information α2 in the detection region D or E, the time until the threshold is reached becomes long, and thus weighting information is set to be large.

A histogram 703 shown in 7C of FIG. 7 corresponds to 303 of 3B of FIG. 3, and indicates a status in which the position of the object 105 deviates downward with respect to the FPD 102. An arrow 720 in the histogram 703 indicates a deviation amount in the lower direction from the central portion 700 of the FPD 102.

Since the ratio of the abdominal part with low radiation transmittance is low in the object structure in the detection region D or E, the number of pixels in the detection region D or E that outputs the monitor signal value corresponding to the reference output information α2 can increase, as shown in the histogram 703.

On the other hand, since the object structure in the detection region does not change significantly in the detection region A, B, or C but the ratio of the object structure with the same radiation transmittance decreases in the detection region A, B, or C, the number of pixels in the detection region A, B, or C that outputs the monitor signal value corresponding to the reference output information α1 can decrease. For example, if the threshold determination processing is performed based on the OR condition (logical sum), the monitor signal value increases, as compared with the assumed reference output information α2 in the detection region D or E, the time until the threshold is reached becomes short, and thus weighting information is set to be small.

The weighting unit 202 may compare, with the histogram 701 assumed as a reference, the histogram 702 or 703 acquired by analyzing the monitor signal value output from each detection region at the time of imaging. Then, the weighting unit 202 may obtain the peak ratio by pattern matching, thereby acquiring a registration error (registration error amount and registration error direction) of the object 105 with respect to each detection region in the FPD 102. If no registration error occurs, the peaks in the histogram 702 or 703 can have the same pattern as that of the peaks in the reference histogram 701. The reference histogram 701 may be stored in, for example, a table shown in FIG. 9 in linkage with the imaging condition information, and the pattern of the histogram 701 may be acquired together with the reference weighting information and the reference output information.

Next, weighting in the radiation imaging apparatus 102 according to the first embodiment will be described next. The weighting unit 202 of the FPD processing unit 200 generates weighting information of each detection region based on comparison between the reference value (reference output information) of the output signal and the monitor signal value output from each detection region. For each detection region, the reference weighting information transmitted from the reference weighting unit 1038 of the imaging control apparatus 103 is set as the initial value of the weighting information of each detection region. The weighting unit 202 changes the reference weighting information set as the initial value based on the weighting information generated based on the comparison between the reference output information and the monitor signal value output from each detection region in real time. The weighting unit 202 sets weighting information representing a weight for each of the plurality of detection regions.

The weighting unit 202 weights, by using the weighting information, the monitor signal value (combined monitor signal value) output from the signal combining unit 201. When performing automatic exposure control (AEC), the weighting unit 202 generates dose information (weighted monitor signal value (combined monitor signal value)) based on the weighting information for each detection region and the monitor signal value (combined monitor signal value) output from the signal combining unit 201. The weighting unit 202 weights the monitor signal value (combined monitor signal value) by calculating the weighting information for the monitor signal value (combined monitor signal value) (multiplying the monitor signal value by the weighting information). Calculation of the weighting information set for each detection region will be described in detail later.

Note that the monitor signal value (combined monitor signal value) used for calculation may be, for example, the average value of the monitor signal values of the detection pixels included in each detection region, or the average value of the monitor signal values of a predetermined number of detection pixels selected from the plurality of detection pixels included in the detection region.

Based on the dose information (weighted monitor signal value) generated by the weighting unit 202 or the weighted average value of the weighted monitor signal values, the threshold determination unit 204 generates determination information for determining whether to continue automatic exposure control (AEC). The weighted average value can be set to a value given by (a*l+b*m+c*n+ . . . +e*p)/(l+m+n+ . . . +p) where a, b, c, . . . , and e represent monitor signal values detected in each detection region, and l, m, n, . . . , and p represent weights corresponding to the monitor signal values in each detection region.

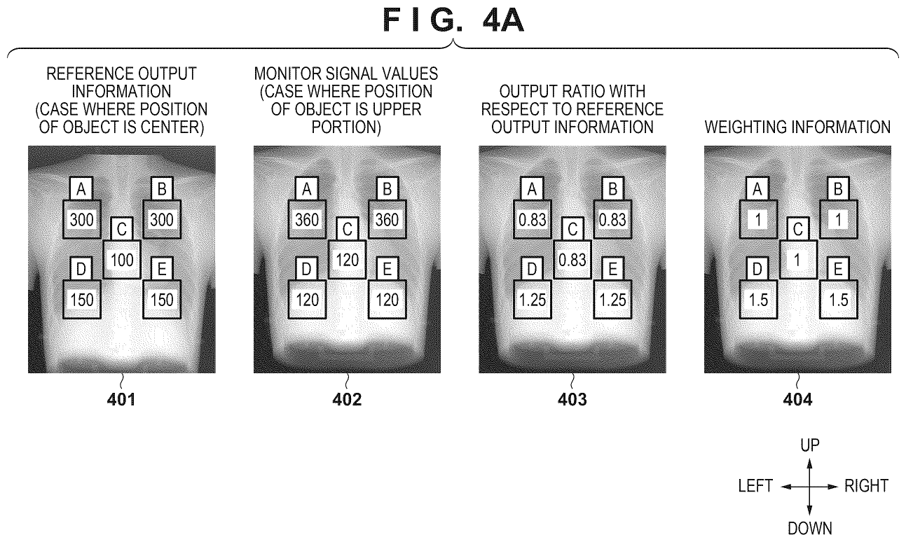

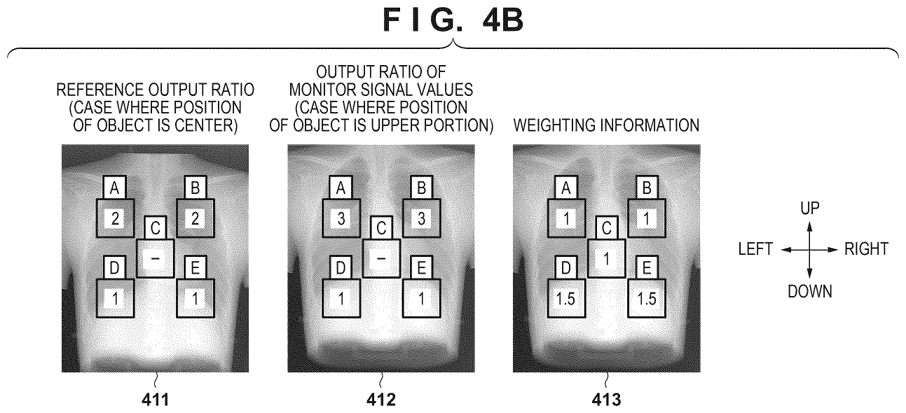

Next, an overview of generation of weighting information using the registration error information of the object 105 in the radiation imaging apparatus 102 and AEC determination according to the first embodiment will be described. FIGS. 4A and 4B are views showing examples of weighting based on dose information in a case where the position of the object 105 deviates upward on the paper surface with respect to the radiation imaging apparatus 102 (FPD) according to the first embodiment. The generation processing of the weighting information based on the dose information in a status in which the position of the object 105 deviates upward on the paper surface with respect to the FPD 102 in imaging of the front of the chest will be exemplified with reference to FIGS. 4A and 4B.

A case where an Exposure Index (EI) value as a dose index value is used as the output information (reference output information) as the reference of the monitor signal value will be exemplified with reference to FIG. 4A. The weighting unit 202 may hold the reference output information of the monitor signal value in the internal memory, or acquire, via the communication IF unit 205, the reference output information of the monitor signal value stored in the storage unit 1036 from the imaging control apparatus 103. The same component as the storage unit 1036 may be provided in the FPD processing unit 200, and may store the table or the information acquired from the imaging control apparatus 103. That is, the FPD processing unit 200 may include an internal storage unit that stores the imaging condition information for capturing a radiation image, and the reference output information and the pieces of reference weighting information for the plurality of detection regions, which are associated with the imaging condition information.

In this example, a case where the position of the object 105 is set at the center of the FPD 102 (a state in which no relative registration error occurs) is set as a reference. The weighting unit 202 generates weighting information set for each detection region by comparing the monitor signal value in each detection region with the output information (reference output information) obtained with reference to the position of the object with respect to the FPD 102 (the plurality of detection regions). For example, the weighting unit 202 may set, as the reference output information of the monitor signal value as a reference, the signal value (signal information) acquired from an image of the same object 105 captured in the past. At this time, for example, the image of the same object 105 captured in the past may be held in the internal memory of the weighting unit 202 or stored in the storage unit 1036 of the imaging control apparatus 103. The weighting unit 202 may acquire image information of the object 105 from the internal memory, or can acquire image information of the same object 105 captured in the past from the imaging control apparatus 103 via the communication IF unit 205.

As shown in a display example 401 of FIG. 4A, the pieces of reference output information of the monitor signal values in the detection regions A to E are 300 in the detection region A (A: 300), 300 in the detection region B (B: 300), 100 in the detection region C (C: 100), 150 in the detection region D (D: 150), and 150 in the detection region E (E: 150).

The weighting unit 202 receives the monitor signal values of the detection regions selected from the plurality of detection regions in the FPD 102. In an example shown in a display example 402 of FIG. 4A, the detection regions A to E are selected detection regions, and the monitor signal values detected in the respective detection regions are 360 in the detection region A (A: 360), 360 in the detection region B (B: 360), 120 in the detection region C (C: 120), 120 in the detection region D (D: 120), and 120 in the detection region E (E: 120).

In a status in which the object 105 deviates upward with respect to the FPD 102, since the ratio of the abdominal part with low radiation transmittance is high in the object structure in the detection region D or E, the monitor signal (D: 120 or E: 120) detected in the detection region D or E decreases, as compared with the reference output information (D: 150 or E: 150), and decreases to 0.8 times of the reference output information.

On the other hand, in the detection region A, B, or C, the object structure in the detection region does not change significantly. However, the monitor signal value (A: 360, B: 360, or C: 120) detected in in the detection region A, B, or C increases, as compared with the reference output information (A: 300, B: 300, or C: 100), and increases to 1.2 times of the reference output information.

Thus, when the monitor signal value in the detection region D or E decreases, the time until the predetermined dose is reached becomes long, and the dose to the object 105 increases. The weighting unit 202 calculates a deviation of the monitor signal value from the reference output information, and calculates a weight for each detection region.

The weighting unit 202 performs an operation of dividing the reference output information by the detected monitor signal value (reference output information/detected monitor signal value), thereby acquiring information (reference output ratio: an output ratio with respect to the reference output information) representing the deviation of the monitor signal value from the reference output information (a display example 403).

As shown in the display example 403 of FIG. 4A, the information (the output ratio with respect to the reference output information) representing the deviation of the monitor signal value from the reference output information is A: 300/360=0.83 in the detection region A, and B: 300/360=0.83 in the detection region B (the display example 403 of FIG. 4A). Furthermore, the information is C: 100/120=0.83 in the detection region C, D: 150/120=1.25 in the detection region D, and E: 150/120=1.25 in the detection region E (the display example 403 of FIG. 4A).

With respect to the detection region A, B, or C where the object structure does not change, the weighting unit 202 sets “1” as a weight for the current monitor signal value, and maintains the current monitor signal value (404 of FIG. 4A).

When the weighting unit 202 obtains the monitor signal value of the detection region D so that the relationship between the monitor signal value of the detection region A (monitor signal value A: 360) and that of the detection region D satisfies a ratio of 2:1 (A (300):D (150)) between the pieces of reference output information, the monitor signal value needed in the detection region D is D: 180. On the other hand, the current monitor signal value in the detection region D is D: 120, and the weighting unit 202 sets a weight “1.5” for the detection region D, which is needed to satisfy the ratio of 2:1 between the pieces of reference output information (404 of FIG. 4A). The weighting unit 202 sets a weight “1.5” for the detection region E, similar to the detection region D (404 of FIG. 4A).

In this case, for example, by multiplying the monitor signal value “360” in the detection region A by the weighting information “1”, the weighting unit 202 acquires the weighted monitor signal value “360”. Similarly, by multiplying the monitor signal value “120” in the detection region D by the weighting information “1.5”, the weighting unit 202 acquires the weighted monitor signal value “180”, and the ratio between the weighted monitor signal values is A (A: 360):D (D: 180)=2:1 is obtained. The ratio (2:1) between the weighted monitor signal values is equal to the ratio (A (300):D (150)=2:1) between the pieces of reference output information (the display example 401 of FIG. 4A). The weighting unit 202 of the FPD processing unit 200 generates weighting information so as to reduce the deviation of the output signal (monitor signal value) from the reference output information.

In the example shown in FIG. 4A, imaging is performed in advance in a state in which the position of the object 105 deviates with respect to the FPD 102, the generated reference output information and reference weighting information are acquired and held in the table shown in FIG. 9. Note that the disclosed technique is not limited to the example shown in FIG. 4A, and can be applied to a case where weighting information of each detection region is calculated during radiation irradiation and the reference weighting information set as the initial value is changed.

The weighting unit 202 of the FPD processing unit 200 acquires the ratio between the output signals (monitor signal values) output from the predetermined detection regions during capturing of a radiation image, and generates weighting information so as to match the ratio between the output signals (monitor signal values) with the ratio between the pieces of reference output information. A case where the ratio (reference output ratio) between the pieces of reference output information in specific detection regions is used to generate weighting information during radiation irradiation will be exemplified with reference to FIG. 4B. The weighting unit 202 sets, as a reference, the ratio (reference output ratio) between the pieces of reference output information based on the pieces of held reference output information. In a display example 411 of FIG. 4B, the reference output ratio in the detection regions A and D is A:D=2:1. The reference output ratio in the detection regions B and E is B:E=2:1.

The weighting unit 202 receives the monitor signal values of the detection regions A to E selected from the plurality of detection regions in the FPD 102, analyzes the monitor signal values of the respective detection regions A to E, and acquires the output ratios between the monitor signal values of the respective detection regions A to E.

As shown in a display example 412 of FIG. 4B, the output ratio between the monitor signal value of 360 in the detection region A (A: 360) and the monitor signal value of 120 in the detection region D (D: 120) is A:D=3:1. Similarly, the output ratio between the monitor signal value of 360 in the detection region B (B: 360) and the monitor signal value of 120 in the detection region E (E: 120) is B:E=3:1.

For example, with respect to the detection regions A and D, the weighting unit 202 compares the output ratio (3:1) between the acquired monitor signal values for the respective detection regions with the output ratio (reference output ratio (2:1)) as a reference between the detection regions. Then, the weighting unit 202 calculates a weight for each detection region so as to obtain the output ratio equal to the output ratio (reference output ratio) as a reference between the detection regions. As shown in a display example 413 of FIG. 4B, the weighting unit 202 sets a weight for each of the detection regions A to E so as to obtain the output ratios equal to the reference output ratio (A:D=B:E=2:1). More specifically, the weighting unit 202 sets the pieces of weighting information for the respective detection regions to A: 1, B: 1, C: 1, D: 1.5, and E: 1.5.

The weighting unit 202 generates dose information (weighted monitor signal value) by performing an operation of “weighting information”*“monitor signal value” for each monitor signal value received from the signal combining unit 201 based on the set weighting information, and transmits the dose information to the threshold determination unit 204.

The threshold determination unit 204 determines, based on the determination method (AND condition, OR condition, AVG condition, or the like) represented by a logical expression, whether the weighted monitor signal value exceeds a threshold (condition). If the weighted monitor signal value exceeds the threshold, the threshold determination unit 204 generates, via the communication IF unit 205, determination information (an irradiation stop signal) representing that the weighted monitor signal value exceeds the threshold, and transmits the determination information to the imaging control apparatus 103 via the communication IF unit 205.

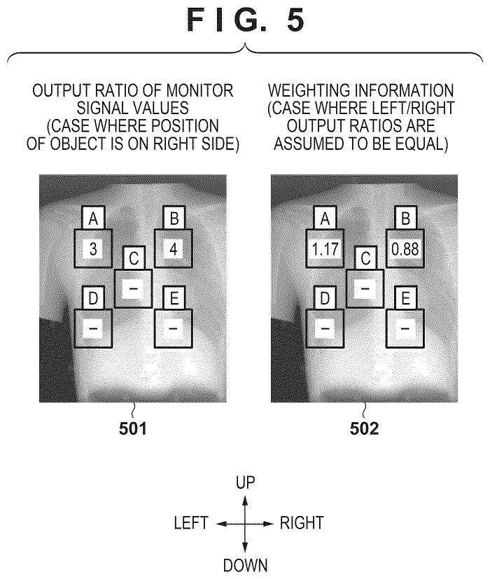

In the examples described with reference to FIGS. 4A and 4B, a case where the weighting unit 202 holds the reference output information has been exemplified. However, the present invention is not limited to this, and the weighting unit 202 can generate weighting information without holding the reference output information. A case where the weighting unit 202 can generate weighting information without holding the reference output information as a reference will be described with reference to FIG. 5. If imaging of an imaging part of the object is performed using the detection regions at positions laterally symmetrical to each other among the plurality of detection regions, the weighting unit 202 of the FPD processing unit 200 generates weighting information so as to obtain an equal ratio between the output signals (monitor signal values) output from the detection regions at the positions laterally symmetrical to each other during capturing of a radiation image.

In the example shown in FIG. 5, in a case of frontal imaging in which an equal output ratio is obtained between the left and right monitor signal values in the left and right detection regions, the weighting unit 202 generates weighting information by assuming that an equal output ratio is obtained between the monitor signal values in the left and right detection regions. The weighting unit 202 can determine an imaging part of the object 105 based on the imaging condition information acquired from the imaging control apparatus 103 via the communication IF unit 205, and determine whether imaging is imaging (frontal imaging) in which an equal output ratio is obtained between the monitor signal values in the left and right detection regions. If imaging is imaging (frontal imaging) in which an equal output ratio is obtained between the monitor signal values in the left and right detection regions, the weighting unit 202 generates weighting information using lateral symmetry.

In a display example 501 shown in FIG. 5, in imaging of the front of the chest, the position of the object 105 deviates rightward with respect to the FPD 102, and the output ratio between the monitor signal values output from the left and right detection regions A and B is A:B=3:4.

The weighting unit 202 acquires an averaged output ratio of 3.5 (=(3+4)/2) between the monitor signal values so that the left and right numbers in the output ratio between the monitor signal values of the left and right detection regions A and B are qual to each other. The averaged output ratio (A:B=3.5:3.5) between the monitor signal values can be the reference output ratio between the monitor signal values in the left and right detection regions. The weighting unit 202 calculates weights for the left and right detection regions A and B so as to obtain an output ratio equal to the averaged output ratio between the monitor signal values. In this case, the weighting unit 202 acquires 1.17 (=3.5/3) as weighting information of the detection region A (a display example 502 of FIG. 5). Furthermore, the weighting unit 202 acquires 0.88 (=3.5/4) as weighting information of the detection region B (the display example 502 of FIG. 5).

A case where an obstacle such as a pacemaker exists in the object 105 and a deviation between a monitor signal value output from a detection region and the reference output information as a reference is extremely large may occur. In this case, a registration error of the object 105 may erroneously be inferred, and weighting information may not be generated correctly. To cope with this, the FPD processing unit 200 may have a function capable of selecting non-execution of weighting. If it is known in advance that an obstacle exists in the object 105, the imaging condition setting unit 1031 may be able to select non-execution of weighting. Information (weighting non-execution information) representing non-execution of weighting is transmitted to the FPD processing unit 200 of the FPD 102 via the communication IF unit 1037. If the weighting non-execution information is received via the communication IF unit 205, the weighting unit 202 of the FPD processing unit 200 can prevent weighting information from being generated.

Alternatively, weighting may not be executed during radiation irradiation in accordance with the analysis result of the monitor signal value. If, for example, the deviation between the monitor signal value output from the detection region and the reference output information as a reference is equal to or larger than a predetermined value, the weighting unit 202 can prevent weighting from being executed. At this time, the weighting unit 202 can transmit information (weighting non-execution information) representing non-execution of weighting to the display control unit 1035 of the imaging control apparatus 103 via the communication IF unit 205, and displays information representing non-execution of weighting on the display unit 106, thereby notifying the operator of it.

### Second Embodiment

The second embodiment of the disclosed technique will be described next. The first embodiment has explained the arrangement in which weighting information is generated using registration error information of the object 105 with respect to the FPD 102. The second embodiment will describe an arrangement in which weighting information is generated using deviation information from standard reference physique information with respect to physique information of an object 105 such as the size and body thickness of the object.

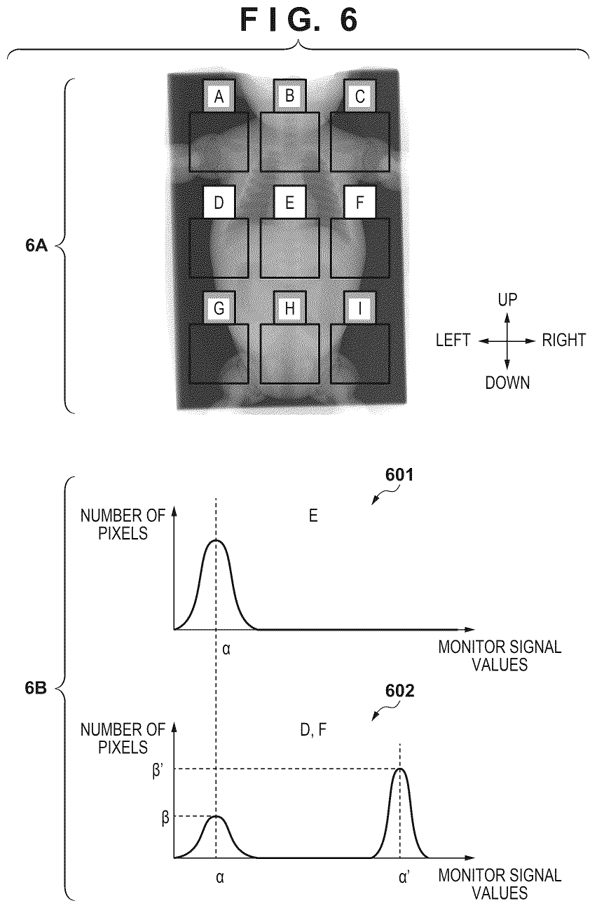

6A of FIG. 6 is a view exemplifying a case where an infant is imaged as an object. A case where a deviation of a monitor signal value occurs with respect to reference output information as a reference due to the influence of a small size of the object, as compared with the standard reference physique information will be described with reference to 6A of FIG. 6. In this example, detection regions of an FPD 102 will be described by exemplifying a case where there exist nine detection regions A to I arranged in 3 rows×3 columns and detection regions selected from the nine detection regions A to I are three detection regions D, E, and F.

If the size of the object 105 is smaller than the standard reference physique information as a reference, X-rays may not be transmitted through the object 105 in the selected detection regions D, E, and F, thereby generating through-exposure regions where X-rays directly enter the FPD 102. For example, no through-exposure region is generated in the detection region E located at the center and through-exposure regions may be generated in the detection regions D and F located on the left and right sides of the detection region E.

A weighting unit 202 of an FPD processing unit 200 compares the distributions of the peaks of output signals (monitor signal values) for the plurality of detection regions to specify the detection region that outputs the output signal (monitor signal value) of the peak larger than reference output information, and then sets weighting information of the specified detection region to be smaller than weighting information for the detection region that outputs the output signal corresponding to the reference information. 6B of FIG. 6 shows graphs each showing a result of executing histogram analysis of the monitor signal values for each detection region. As shown in 601 of 6B of FIG. 6, in the detection region E where no through-exposure region is generated, the peak hardly deviates with respect to reference output information α. On the other hand, as shown in 602 of 6B of FIG. 6, in each of the detection regions D and F where a through-exposure region is generated, the peak of output information α′ of the monitor signal values of the detection region where a through-exposure region is generated is larger than the reference output information α. That is, the peak deviates with respect to the reference output information α. To cope with this, the weighting unit 202 can reduce the weight for each of the detection regions D and F, thereby reducing the influence of generation of a through-exposure region on AEC determination (determination of whether a predetermined cumulative dose is reached).

In this example, if the monitor signal value is an average value in the detection region, the weighting unit 202 can acquire a monitor signal value M (D, E) in the detection region D or F by:

$$monitor signal value M ( D , E ) \approx α + ( {α}^{′} - α ) \times {β}^{′} / ( β + {β}^{′} ) \qquad ( 1 )$$

where α represents output information (reference output information) as a reference, and α′ represents output information of the monitor signal value in the detection region where a through-exposure region is generated. β represents the number of pixels of the detection region that outputs the monitor signal value corresponding to the reference output information α, and β represents the number of pixels of the detection region that outputs the monitor signal value α′, where a through-exposure region is generated.

The weighting unit 202 need only set weighting information so that the average value M of the monitor signal values in the through-exposure region becomes close to the reference output information α. In this case, the weighting unit 202 can acquire weighting information using the monitor signal value M (D, E) obtained by equation (1) above, given by:

$$weighting information = α / M ( D , E ) \qquad ( 2 )$$

The type of the monitor signal value used for AEC threshold determination may be changed. For example, in AEC threshold determination, if it is set to use the maximum value of the monitor signal values in the selected detection region, the type of the monitor signal value may be changed to use the minimum value of the monitor signal values in the selected detection region at a timing when it is known, as a result of histogram analysis, that the influence of the through-exposure region is large. Alternatively, the logical operation of comparing the monitor signal value with a threshold for determining AEC may be changed.

An example in which the size of the object 105 is different from the standard reference physique information in the left-and-right direction of the FPD 102 has been described with reference to 6A and 6B of FIG. 6 but the present invention is not limited to this. The disclosed technique can similarly be applied to a case where the body thickness of the object 105 is different from the standard reference physique information.

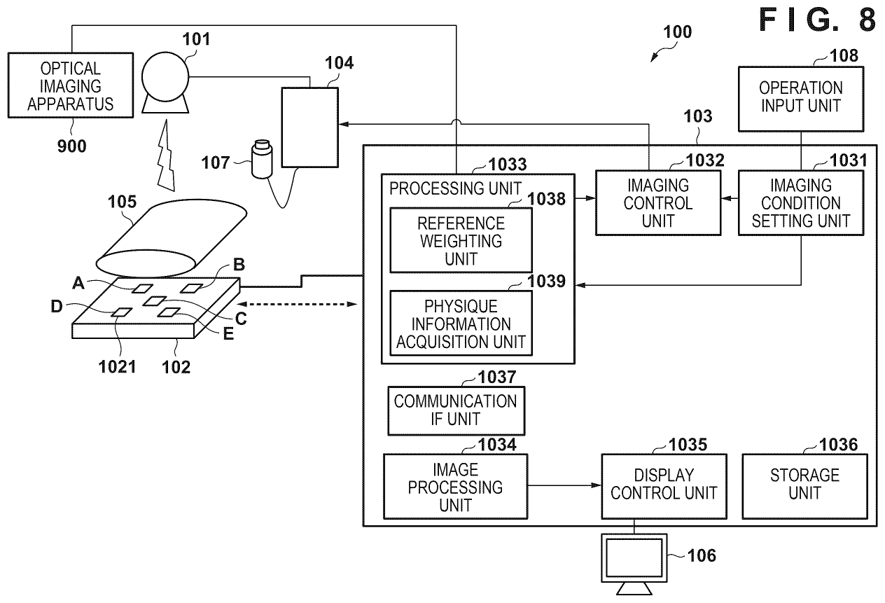

FIG. 8 is a view showing an example of the configuration of a radiation imaging system 100 including the radiation imaging apparatus 102 according to the second embodiment. The basic system configuration is the same as that of the radiation imaging system 100 described in the first embodiment, and an imaging control apparatus 103 and the radiation imaging apparatus 102 that captures a radiation image based on radiation emitted from a radiation source 101 are provided. The imaging control apparatus 103 is connected to the radiation imaging apparatus 102 and a radiation control apparatus 104 that controls the radiation source 101 by, for example, a wired or wireless network or dedicated lines, and controls radiation imaging using the radiation imaging apparatus 102 and the radiation source 101.

The radiation imaging system 100 according to the second embodiment is provided with an optical imaging apparatus 900 (for example, an optical camera) that captures an optical image of the object 105. As a function component of a processing unit 1033, a physique information acquisition unit 1039 is added. For example, the optical imaging apparatus 900 may be arranged near the radiation source 101. The optical imaging apparatus 900 is not limited to a single optical imaging apparatus 900, and a plurality of optical imaging apparatuses 900 may be used. The optical imaging apparatus 900 acquires an optical image of the object 105 before performing radiation imaging. The optical image acquired by the optical imaging apparatus 900 is transmitted to the processing unit 1033.

The physique information acquisition unit 1039 acquires physique information of the object based on the optical image of the object captured by the optical imaging apparatus 900, and acquires a deviation from the reference physique information as a reference. The physique information acquisition unit 1039 extracts physique information representing the characteristics (for example, the body width and body thickness) of the physique of the object 105 by performing image processing of extracting an outline for the optical image acquired by the optical imaging apparatus 900. The physique information acquisition unit 1039 acquires the difference between the reference physique information and the physique information of the object 105 by comparing the acquired physique information of the object 105 with the standard reference physique information corresponding to the age, sex, and the like of the object 105.

If the acquired difference has a value falling within a predetermined range, a reference weighting unit 1038 may determine that the reference physique information matches the physique information of the object 105. On the other hand, if the acquired difference falls outside the predetermined range, the reference weighting unit 1038 determines that the physique information of the object 105 is different from the reference physique information, and changes, based on the difference, the reference output information and reference weighting information set for each detection region.

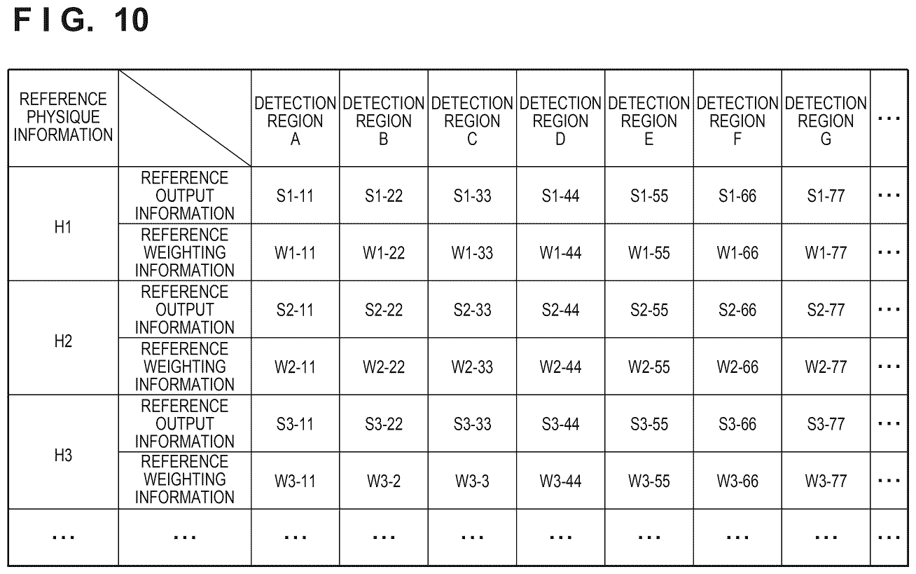

FIG. 10 is a view exemplifying a table that stores reference physique information, reference output information, and reference weighting information corresponding to the age, sex, and the like of the object 105 stored in the storage unit 1036. In the table, the reference value (reference output information) of the output signal in each detection region and reference weighting information in each detection region are set for each piece of reference physique information.

For example, if the physique information of the object 105 is different from reference physique information H1, the reference weighting unit 1038 changes, based on the difference between the reference physique information H1 and the physique information of the object 105, the reference output information (S1-11, S1-22, . . . ) and the reference weighting information (W1-11, W1-22, . . . ) set for each detection region so as to reduce the value of the difference between the pieces of physique information. The reference weighting unit 1038 acquires the changed reference output information and reference weighting information as initial values, and transmits them to the FPD 102 via the communication IF unit 1037. The weighting unit 202 in the FPD processing unit 200 sets, as reference values in each detection region, the reference output information and the reference weighting information transmitted from the reference weighting unit 1038, and captures a radiation image of the object 105.

According to this embodiment, even if the physique information of the object 105 is different from the standard reference physique information, the reference output information and the reference weighting information in each detection region can be changed in consideration of the difference of the physique information of the object 105. This can suppress the variation of the dose of radiation in automatic exposure control.

### Third Embodiment

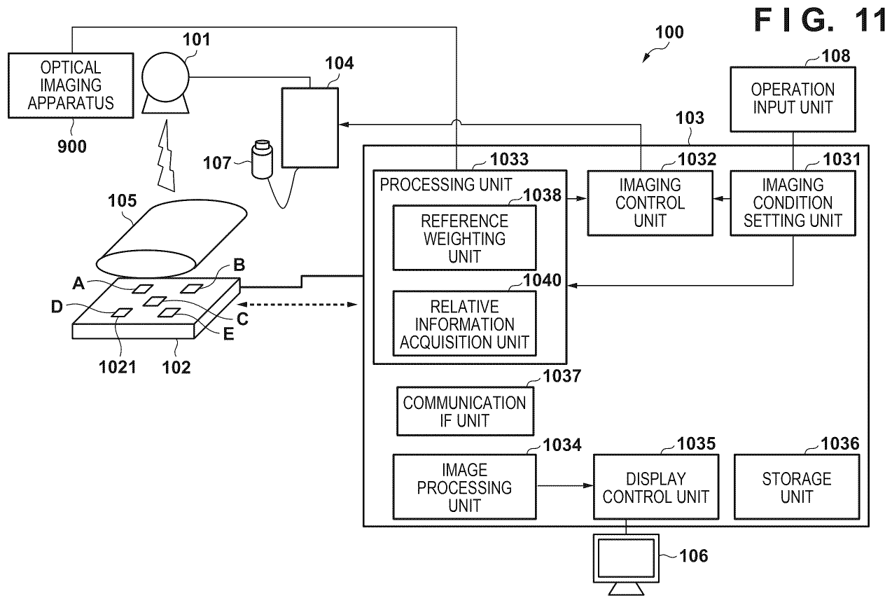

FIG. 11 is a view showing an example of the configuration of a radiation imaging system 100 including a radiation imaging apparatus 102 according to the third embodiment. The basic system configuration is the same as that of the radiation imaging system 100 described in the first embodiment, and an imaging control apparatus 103 and the radiation imaging apparatus 102 that captures a radiation image based on radiation emitted from a radiation source 101 are provided. The imaging control apparatus 103 is connected to the radiation imaging apparatus 102 and a radiation control apparatus 104 that controls the radiation source 101 by, for example, a wired or wireless network or dedicated lines, and controls radiation imaging using the radiation imaging apparatus 102 and the radiation source 101.

The radiation imaging system according to the third embodiment is provided with an optical imaging apparatus 900 (for example, an optical camera) that captures an optical image of an object 105. As a function component of a processing unit 1033, a relative information acquisition unit 1040 is added. For example, the optical imaging apparatus 900 may be arranged near the radiation source 101. The optical imaging apparatus 900 acquires an optical image of the object 105 before performing radiation imaging. The optical image acquired by the optical imaging apparatus 900 is transmitted to the processing unit 1033.

The relative information acquisition unit 1040 acquires a registration error of the object with respect to a plurality of detection regions using the optical image of the object captured by the optical imaging apparatus 900. By performing image processing for the optical image acquired by the optical imaging apparatus 900, the relative information acquisition unit 1040 acquires a skeleton model of the object 105 that is obtained by extracting the outline of the body shape of the object 105. The relative information acquisition unit 1040 calculates the positional relationship among the respective detection regions of the FPD 102 from the skeleton model and optical image of the object 105. Then, the relative information acquisition unit 1040 calculates relative registration error information with respect to each detection region by comparing the positional relationship calculated from the skeleton model and optical image with the ideal positional relationship between the object and each detection region that is held in advance. Before capturing a radiation image of the object 105, the relative information acquisition unit 1040 acquires a relative registration error with respect to each detection region in the FPD 102 based on the optical image of the object 105. The acquired relative registration error includes, for example, a status in which the position of the object 105 deviates upward on the paper surface with respect to the FPD 102, as described in 302 of 3A of FIG. 3 and a status in which the position of the object 105 deviates downward on the paper surface with respect to the FPD 102, as described in 303 of 3B of FIG. 3. Note that the registration error is not limited to a registration error in the up-and-down direction and includes an error in the left-and-right direction. The ideal positional relationship between the detection region and the object includes, for example, a case where the object 105 is located at the center of the FPD 102, as described using 301 of 3A of FIG. 3.

An imaging condition setting unit 1031 accepts imaging condition information input via an operation input unit 108 by an operator, and transmits the received imaging condition information to an imaging control unit 1032 and the processing unit 1033.

If no relative registration error occurs (the ideal positional relationship is obtained), a reference weighting unit 1038 of the processing unit 1033 sets reference output information and reference weighting information for the detection region in the FPD 102 based on the imaging condition information set by the imaging condition setting unit 1031.

If there exist a plurality of detection regions in the pixel region of the FPD 102, the reference weighting unit 1038 sets reference output information and reference weighting information for each detection region. In a case where no registration error occurs, the reference weighting unit 1038 acquires reference output information and reference weighting information from, for example, a table stored in a storage unit 1036.

On the other hand, in a case where a relative registration error is acquired (a registration error occurs), the reference weighting unit 1038 of the processing unit 1033 changes, based on the relative registration error information, the reference output information and the reference weighting information for the detection region in the FPD 102.

For example, in a case of imaging condition information D1, if a relative registration error of the object 105 with respect to the FPD 102 is acquired, the reference weighting unit 1038 changes reference output information (S1-1, S1-2, . . . ) and reference weighting information (W1-1, W1-2, . . . ) set for each detection region in correspondence with the imaging condition information D1, so as to reduce a relative registration error amount. The reference weighting unit 1038 acquires the changed reference output information and reference weighting information as initial values, and transmits them to the FPD 102 via the communication IF unit 1037.

A weighting unit 202 in an FPD processing unit 200 sets, as reference values in each detection region, the reference output information and the reference weighting information transmitted from the reference weighting unit 1038, and captures a radiation image of the object 105. The FPD processing unit 200 generates determination information for controlling irradiation with radiation by comparing, with a preset threshold, dose information obtained by weighting an output signal (monitor signal value) based on the changed weighting information.

According to this embodiment, even if a relative registration error of the object 105 occurs with respect to each detection region of the FPD 102, it is possible to change the reference output information and the reference weighting information in each detection region in consideration of the relative registration error amount between each detection region of the FPD 102 and the position of the object 105. This can suppress the variation of the dose of radiation in automatic exposure control.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2023-216196, filed Dec. 21, 2023 which is hereby incorporated by reference herein in its entirety.

What is claimed is:

1. A radiation imaging apparatus that includes a plurality of receptor fields where a dose of radiation capable of being detected during radiation imaging in which a radiation irradiation apparatus emits the radiation and is capable of executing processing for stopping the irradiation based on a result of the dose detection in at least one of the plurality of receptor fields, the radiation imaging apparatus comprising:
one or more controllers configured to adjust a parameter associated with the receptor field used for the dose detection based on registration error information of an object at the time of the radiation imaging.

2. The apparatus according to claim 1, wherein the one or more controllers
process output signals output from a plurality of detection regions that are provided in a pixel region where a plurality of pixels each for detecting the radiation are arrayed and include detection pixels each for outputting a signal corresponding to the dose of the radiation,
generate weighting information for each of the plurality of detection regions by comparing the output signal with preset reference information, and
generate determination information for controlling the irradiation with the radiation by comparing, with a preset threshold, dose information obtained by weighting the output signal based on the weighting information.

3. The apparatus according to claim 2, wherein
the reference information includes reference output information output from each of the plurality of detection regions in a case where no registration error of the object occurs with respect to the plurality of detection regions, and
the one or more controllers generate the weighting information so as to reduce a deviation of the output signal with respect to the reference output information.

4. The apparatus according to claim 3, wherein the one of more controllers include a storage memory configured to store imaging condition information for capturing a radiation image, and the reference output information and reference weighting information for each of the plurality of detection regions, which are associated with the imaging condition information.

5. The apparatus according to claim 4, wherein the one or more controllers
acquire, from the storage memory, the reference output information and the reference weighting information which are associated with the imaging condition information, and
change, based on the weighting information generated during capturing of the radiation image, the reference weighting information set as an initial value before capturing the radiation image.

6. The apparatus according to claim 3, wherein
the reference information includes a ratio between the pieces of reference output information in predetermined detection regions among the plurality of detection regions,
the one or more controllers acquire a ratio between the output signals output from the predetermined detection regions during capturing of the radiation image, and
generate the weighting information so as to match the ratio between the output signals with the ratio between the pieces of reference output information.

7. The apparatus according to claim 2, wherein the one or more controllers generate the weighting information for a detection region selected from the plurality of detection regions.

8. The apparatus according to claim 2, wherein in a case where imaging of an imaging part of the object is performed using detection regions located at positions laterally symmetrical to each other among the plurality of detection regions, the one or more controllers generate the weighting information so as to obtain an equal ratio between the output signals output from the detection regions at the positions laterally symmetrical to each other during capturing of a radiation image.

9. The apparatus according to claim 2, wherein the one or more controllers
compare distributions of peaks of the output signals for the plurality of detection regions,
specify a detection region that outputs the output signal of the peak larger than the reference information, and
set weighting information of the specified detection region to be smaller than weighting information for the detection region that outputs the output signal corresponding to the reference information.

10. The apparatus according to claim 2, wherein in a case where a deviation between the output signal and the reference information is not smaller than a predetermined value, the one or more controllers do not generate the weighting information.

11. The apparatus according to claim 5, wherein the one or more controllers set, as the initial value, weighting information obtained by changing the reference weighting information, based on a registration error of the object with respect to the plurality of detection regions acquired using an optical image of the object.

12. The apparatus according to claim 4, wherein the one or more controllers set, as the initial value, weighting information obtained by changing the reference weighting information, based on a deviation between preset reference physique information and physique information of the object acquired using an optical image of the object.

13. The apparatus according to claim 1, wherein the one or more controllers
process output signals output from a plurality of detection regions that are provided in a pixel region where a plurality of pixels each for detecting the radiation are arrayed and include detection pixels each for outputting a signal corresponding to the dose of the radiation,
generate weighting information for each of the plurality of detection regions by comparing the output signal with preset reference information, and
generate determination information for controlling the irradiation with the radiation by comparing, with a preset threshold, dose information obtained by weighting the output signal based on the weighting information.

14. The apparatus according to claim 1, wherein the one or more controllers
acquire physique information of the object based on an optical image of the object captured by an optical imaging unit, and acquire a deviation from reference physique information as a reference,
change weighting information as a reference based on the deviation of the physique information,
process output signals output from a plurality of detection regions that are provided in a pixel region where a plurality of pixels each for detecting the radiation are arrayed and include detection pixels each for outputting a signal corresponding to the dose of the radiation, and
control the irradiation with the radiation by comparing, with a preset threshold, dose information obtained by weighting the output signal based on the changed weighting information.

15. The apparatus according to claim 1, wherein the one or more controllers
acquire a registration error of the object with respect to the plurality of detection regions using an optical image of the object captured by an optical imaging unit,
change weighting information as a reference based on the registration error,
process output signals output from a plurality of detection regions that are provided in a pixel region where a plurality of pixels each for detecting the radiation are arrayed and include detection pixels each for outputting a signal corresponding to the dose of the radiation, and
generate determination information for controlling irradiation with the radiation by comparing, with a preset threshold, dose information obtained by weighting the output signal based on the changed weighting information.

16. The apparatus according to claim 1, wherein the one or more controllers
acquire dose distribution information based on first dose detection information acquired at a first timing during radiation irradiation after a start of the irradiation with the radiation by the radiation irradiation apparatus,
update weighting information concerning the receptor field used for the dose detection based on the dose distribution information corresponding to the registration error information, and
perform communication for stopping the irradiation based on second dose detection information acquired at a second timing during the radiation irradiation, the updated weighting information, and threshold information of the receptor field used for the dose detection.

17. The apparatus according to claim 1, wherein the one or more controllers
acquire an optical image of the object at a start of radiation imaging or after a start of radiation imaging,
update weighting information concerning the receptor field used for the dose detection based on the optical image corresponding to the registration error information, and
perform communication for stopping the irradiation based on second dose detection information acquired at a second timing during the radiation irradiation, the updated weighting information, and threshold information of the receptor field used for the dose detection.

18. A radiation imaging method of a radiation imaging apparatus that includes a plurality of receptor fields where a dose of radiation capable of being detected during radiation imaging in which a radiation irradiation apparatus emits the radiation and is capable of executing processing for stopping the irradiation based on a result of the dose detection in at least one of the plurality of receptor fields, the radiation imaging method comprising:
adjusting a parameter associated with the receptor field used for the dose detection based on registration error information of an object at the time of the radiation imaging.

19. A non-transitory computer-readable storage medium storing a program for causing a computer to execute a radiation imaging method defined in claim 18.
